(19) United States
(12) Patent Application Publication
(10) Pub. No.: US 2022/0362478 A1
(43) Pub. Date: Nov. 17, 2022

(54) CARTRIDGE SYSTEM FOR RECEIVING A DOSE SENSING MODULE

(71) Applicant: Novo Nordisk A/S, Bagsvaerd (DK)

(72) Inventor: Janus Helbo, Koebenhavn N (DK)

(21) Appl. No.: 17/767,322

(22) PCT Filed: Oct. 8, 2020
(86) PCT No.: PCT/EP2020/078308

§ 371 Date: Apr. 7, 2022

(30) Foreign Application Priority Data
Oct. 8, 2019 (EP) 19201824.0
Aug. 26, 2020 (EP) PCT/EP2020/073871

(51) Int. Cl. A61M 5/315; A61M 5/24

(52) U.S. Cl. CPC A61M 5/31546; A61M 5/31515; A61M 5/31568; A61M 5/24

(57) ABSTRACT

The present invention provides a cartridge system (30) for a drug delivery device, comprising a drug cartridge (20) comprising a cartridge body (21) extending along a reference axis between a distal outlet end portion and a proximal rim (21.2), and a displaceable piston (22) arranged in the cartridge body (21) an axial distance from the proximal rim (21.2), an outer cavity (29) thus being formed between the displaceable piston (22) and the proximal rim (21.2), and a guide element (60, 90) comprising a main guide body (61, 91), and a rim interface member (62, 92) adapted to abut or engage the proximal rim (21.2) and thereby cover the proximal rim (21.2) at least partially. The main guide body (61, 91) extends between a first main guide body end (61.1) bordering the rim interface member (62, 92) and a second main guide body end (61.2) and defines a passage for a sensor unit.

### FIELD OF THE INVENTION

The present invention relates generally to drug delivery devices having integrated dose capturing means, and more specifically to cartridge systems for such drug delivery devices.

### BACKGROUND OF THE INVENTION

Injection devices, such as injection pens, are widely used for self-administration of liquid drugs by people in need of therapeutic treatment. Many injection devices are capable of repeatedly setting and injecting either a fixed or a variable volume of drug upon operation of respective dose setting and dose expelling mechanisms in the device. Some injection devices are adapted to be loaded with a prefilled drug reservoir containing a volume of drug which is sufficient to provide for a number of injectable doses. When the reservoir is empty, the user replaces it with a new one and the injection device can thus be used again and again. Other injection devices are prefilled when delivered to the user and can only be used until the drug reservoir has been emptied, after which the whole injection device is discarded. The various injection devices typically expel the drug by advancing a piston in the reservoir using a motion-controlled piston rod.

Within some therapy areas the tendency of a patient to adhere to the prescribed therapy is dependent on the simplicity of the specific treatment regimen. For example, many people with type 2 diabetes are diagnosed with the disease at a relatively high age where they are less prone to accept a treatment that intervenes too much with their normal way of living. Most of these people do not like to be constantly reminded of their disease and, as a consequence, they do not want to be entangled in complex treatment patterns or waste time on learning to operate cumbersome delivery systems. In essence, many are of the opinion that the less manual involvement the better.

For a person with diabetes it is important to timely administer one or more glucose regulating agents to maximise the time spent in normoglycemia. In that connection, in order to establish an overview of one's adherence to a particular treatment regimen, it is significant to keep track of both when such a regulating agent is administered and how much is administered. Accordingly, it is recommended that the person keeps a log of administered dose sizes and times of administration.

Previously, the establishment and maintenance of such a log would require manually noting down the data, e.g. on paper or a pc. However, as this would entail frequent active involvement many people neglected the importance of establishing the overview. In recognition of this undesirable situation various solutions have been suggested for automatic capturing of the relevant information from the individual injection devices.

For example, WO 2018/078178 (Novo Nordisk A/S) discloses a pen type injection device having a sensor arranged on a deflectable exterior surface of the injection device housing. The deflectable exterior surface is configured to undergo a deflection at a specific angular displacement of an interior component rotationally locked to the piston rod, and the sensor is adapted to output a signal in response to a detected deflection, the signal thus being representative of the angular displacement of the piston rod. Since the amount of drug expelled by the disclosed injection device correlates with the total angular displacement of the piston rod relative to the housing the output signals are automatically captured by a processor in the injection device and used as a basis for an estimation of the administered dose. In addition, the processor may establish a time for reception of the output signals and provide a time stamp for the dose expelling event. The data may then be retrieved via an electronic display on the injection device or by wireless transmission to an external device e.g. having, or being connectable to, a display.

An alternative dose detection solution is presented in WO 2014/128155 (Novo Nordisk A/S) which discloses a pen-type drug delivery device with a fully integrated sensor unit in the form of a piston washer module arranged between the piston rod of the dose expelling mechanism and the cartridge piston. The sensor unit operates like a rotary encoder and comprises a first sensor part which is engaged with the piston rod and a second sensor part which is engaged with the cartridge piston. The relative angular displacement between the two sensor parts exhibited during a dose expelling event, when the piston rod rotates relative to the drug delivery device housing and the cartridge, is detected galvanically and translated to an estimate of the size of the administered dose.

Conventionally, the cartridges used for such drug delivery devices comprise a hollow cartridge body made of glass and having a generally cylindrical main body portion with a proximal rim forming a proximal opening, and a distal narrowing forming an outlet end portion, which is sealed by a penetrable septum. In a drug-filled cartridge the cartridge piston is arranged sealingly in the main body portion, typically a short distance from the proximal opening, whereby an outer cavity is formed between the piston and the proximal opening.

During manufacturing of the drug delivery device disclosed in WO 2014/128155 the sensor unit will be placed in the outer cavity. However, as the proximal rim constitutes the most fragile portion of the cartridge structure and is the part of the cartridge which most frequently exhibits crack formation, it is important, in order to avoid fracture, that it is not accidentally impacted by a hard surface when the distal most portion of the sensor unit is lead through the proximal opening. This places severe demands on the radial alignment of the sensor unit with the proximal opening and, resultantly, on the tolerances in the assembly setup.

### SUMMARY OF THE INVENTION

It is an object of the invention to eliminate or reduce at least one drawback of the prior art, or to provide a useful alternative to prior art solutions.

In particular, it is an object of the invention to provide a solution which prevents a potentially damaging collision between the sensor unit and the proximal rim of the cartridge during assembly.

It is another object of the invention to provide a solution which allows for some degree of slack in the assembly line without increasing the risk of the sensor unit impacting the proximal rim of the cartridge.

In the disclosure of the present invention, aspects and embodiments will be described which will address one or more of the above objects and/or which will address objects apparent from the following text.

In one aspect the invention provides a cartridge system as defined in claim 1.

Accordingly, a cartridge system for use in a drug delivery device is provided, comprising a drug cartridge and a guide element adapted to be arranged in axial extension of one another. The drug cartridge, which is suitable for holding a volume of e.g. liquid drug, comprises a cartridge body having a main body portion, a distal outlet end portion and a proximal rim. A displaceable piston is arranged in the cartridge body an axial distance from the proximal rim, whereby an outer cavity is formed between the displaceable piston and the proximal rim. This outer cavity is destined to become deeper as the displaceable piston is displaced axially in the drug cartridge during use. The guide element, which may be formed, e.g. moulded, as a single piece component or composed of two separately produced parts, comprises a main guide body, and a rim interface member which is adapted to abut or engage the proximal rim and thereby cover the proximal rim at least partially. The main guide body extends between a first main guide body end bordering the rim interface member and a second main guide body end and defines a passage for a sensor unit.

By arranging the guide element such that the rim interface member covers the proximal rim at least partially the guide element allows for insertion of the sensor unit into the outer cavity without the risk of a damaging impact to the proximal rim. The risk of cartridge fracture during assembly of the drug delivery device is thus markedly reduced.

The main guide body may comprise an interior guide surface configured to guide the sensor unit into the outer cavity. In particular embodiments of the invention the interior guide surface tapers radially towards the first main guide body end. Thereby, the main guide body exhibits an interior funnel shape, where the second main guide body end has a larger transversal interior dimension than the first main guide body end. The sensor unit does thus not need to be strictly aligned with the proximal opening initially during insertion into the outer cavity, because the funnel shaped guide surface will guide a radially offset sensor unit into the right radial position as the sensor unit approaches the cartridge. This solution can thus accommodate a certain degree of slack in the assembly setup. p For example, the first main guide body end may exhibit an internal first end diameter which corresponds, at least substantially, to the internal diameter of the proximal rim, and the second main guide body end may exhibit an internal second end diameter which is 5-20% larger, such as 10-15% larger, than the internal first end diameter.

The sensor unit may comprise a distal module part having one or more radially outwardly projecting studs adapted to interface with an interior surface of the cartridge body for impeding relative rotation between the distal module part and the cartridge. The diameter of the distal module part may be only slightly smaller than the internal diameter of the proximal rim, and the radially outwardly projecting studs may be radially inwardly displaceable against a bias force to allow passage through the proximal opening. Hence, the radially outwardly projecting studs may be adapted to transition from an unstrained state to a strained state as the distal module part enters the outer cavity.

The main guide body may, alternatively or additionally, comprise a plurality of splines extending axially between the first main guide body end and the second main guide body end, and a plurality of intermediate keyways formed by the plurality of splines, where each of the plurality of splines comprises a radially facing surface for guiding the sensor unit into the outer cavity.

The splines will thus serve to lead the distal module part axially into the outer cavity, while the keyways provide room for the radially outwardly projecting studs.

In particular embodiments of the invention each of the plurality of splines tapers radially towards the second main guide body end. The radially facing surfaces of the plurality of splines thus together provide a funnel shape for guiding even a radially offset sensor unit securely into the outer cavity, similarly to what is described above.

Each of the plurality of splines may, alternatively or additionally, taper circumferentially towards the second main guide body end, i.e. each intermediate keyway may be wider at the second main guide body end than at the first main guide body end. This allows for greater flexibility in the initial positioning of the sensor unit, as the angular orientation of the distal module part relative to the guide element when the sensor unit is to be inserted into the main guide body is less critical. The main guide body simply accepts the distal module part in a larger number of relative angular positions of the guide element and the sensor unit because the wide keyways at the second main guide body end provide wider entrance sections for the one or more radially outwardly projecting studs.

The rim interface member may comprise a circumferential collar adapted to surround a proximal exterior end portion of the cartridge body. The circumferential collar may have alternating convexly and concavely shaped sections. The convexly shaped sections may follow the contour of the cartridge body and the concavely shaped sections may comprise contact areas which abut the cartridge body to provide for firm attachment of the rim interface member to the drug cartridge.

The circumferential collar may, alternatively or additionally, comprise a plurality of collar partitions, and each collar partition may be circumferentially spaced apart from a neighbouring collar partition to thereby provide respective collar openings therebetween. The collar openings may allow for reception of e.g. radial protrusions of a non-rotatable component to thereby ensure complete rotational fixation of the guide element in the drug delivery device.

Since the drug delivery device may end up shelved for a significant period of time before being taken into use it may be undesirable to install the sensor unit in a position where the radially outwardly projecting studs are in the strained state, as this could lead to a gradual reduction of the contact force applied to the interior surface of the cartridge body over time and resultantly to a gradual loss of friction in the interface between the cartridge body and the distal module part. Consequently, the sensor unit may be installed in a pre-use position where the radially outwardly projecting studs are in the unstrained state, e.g. within the main guide body just outside the proximal opening. In that case the splines furthermore serve to prevent rotation of the distal module part in a pre-use state of the drug delivery device, and thereby to prevent erroneous sensor readings resulting from the drug delivery device e.g. being dropped or otherwise subjected to jolting motion. The sensor unit is then adapted to be moved axially before the first dose expelling, from the pre-use position in which each of the one or more radially outwardly projecting studs is accommodated between two splines to an in-use position in which the one or more radially outwardly projecting studs are in contact with the interior surface of the cartridge wall.

In particular embodiments of the invention the cartridge system further comprises a cartridge holder for accommodating the drug cartridge, and the cartridge holder comprises a radially inwardly extending protrusion adapted to be received in one of the collar openings, thereby rotationally interlocking the cartridge holder and the guide element. In these embodiments a complete rotational fixation of the guide element in the drug delivery device can be ensured by rotational fixation of the cartridge holder to a housing of the drug delivery device.

Each collar partition may comprise at least one convexly shaped section and at least one concavely shaped section. A symmetrical contact interface between the rim interface member and the cartridge body can thereby be established, reinforcing the attachment of the guide element to the drug cartridge. Further, the resulting curved shape of each collar partition will serve as a shock-absorber, protecting the proximal exterior end portion of the cartridge body, including the proximal rim, in case the drug delivery device is dropped to one side. In particular embodiments thereof, each collar partition comprises two convexly shaped sections separated by one concavely shaped section.

The sensor unit is gradually advanced in the drug cartridge as a dose is expelled. Thus, the small radial dimensions of the cartridge body, and the drug delivery device itself, demands a small-sized sensor unit. The constituent mechanical and electrical components do, however, take up some space, and the incorporation of the sensor unit between the piston rod and the displaceable piston accordingly results in the proximal rim taking up a different axial position in the drug delivery device than it would otherwise do.

The guide element may further comprise a plurality of axially compressible flange members extending axially, specifically proximally, from the second main guide body end. These flange members may, by virtue of their shape and/or the material they are made of, act as compression springs in response to an axial impact to the guide element. Accordingly, the flange members may protect the cartridge body in case the drug delivery device is dropped and lands on either end.

In particular embodiments of the invention the plurality of axially compressible flange members constitutes two axially compressible flange members arranged diametrically opposite one another. Free space is thereby provided between them for allowing radially inwards deflection of portions of the cartridge holder during assembly of the cartridge holder and the housing. Such deflections could e.g. occur in connection with a snap fitting of the cartridge holder to the housing as flexible portions of the cartridge holder pass respective snap geometries on interior surface portions of the housing.

In another aspect the invention provides a guide element for use in a cartridge system as described above.

In a further aspect the invention provides a drug delivery device comprising a cartridge system as described above.

The drug delivery device may further comprise a housing, a dose expelling mechanism comprising an axially advanceable piston rod, and a sensor unit for determining a size of an expelled dose, arranged at least partially in the outer cavity and comprising a proximal module part rotationally locked with respect to the axially advanceable piston rod and a distal module part abutting the displaceable piston.

In particular embodiments of the invention the drug delivery device comprises a cartridge system as described above, in which the guide element comprises a plurality of axially compressible flange members, e.g. exactly two axially compressible flange members arranged diametrically opposite one another, extending axially from the second main guide body end, a housing, a dose expelling mechanism comprising an axially advanceable piston rod, and a sensor unit for determining a size of an expelled dose, arranged at least partially in the outer cavity and comprising a proximal module part rotationally locked with respect to the axially advanceable piston rod and a distal module part abutting the displaceable piston,

wherein each of the plurality of axially compressible flange members abuts a transversally extending interior portion of the housing, or a transversally extending structure axially fixed with respect to the housing.

The drug cartridge is thus elastically supported at its proximal end and thereby protected in case the drug delivery device is accidentally dropped and lands on either end.

In other embodiments of the invention the drug delivery device comprises a cartridge system as described above, including the cartridge holder comprising the radially inwardly extending protrusion adapted to be received in one of the collar openings, thereby rotationally interlocking the cartridge holder and the guide element, a housing, a dose expelling mechanism comprising an axially advanceable piston rod, and a sensor unit for determining a size of an expelled dose, arranged at least partially in the outer cavity and comprising a proximal module part rotationally locked with respect to the axially advanceable piston rod and a distal module part abutting the displaceable piston, wherein the cartridge holder further comprises a radially outwardly extending protrusion adapted to engage with the housing, thereby rotationally interlocking the cartridge holder and the housing.

For the avoidance of any doubt, in the present context the term “drug” designates a medium which is used in the treatment, prevention or diagnosis of a condition, i.e. including a medium having a therapeutic or metabolic effect in the body. Further, the terms “distal” and “proximal” denote positions at or directions along a drug delivery device, or a needle unit, where “distal” refers to the drug outlet end and “proximal” refers to the end opposite the drug outlet end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 shows a dose sensing module according to the prior art,

FIG. 2 is a perspective longitudinal section view of an injection device with a cartridge system according to an exemplary embodiment of the invention,

FIG. 3 is an exploded view of a dose sensing module in the injection device,

FIG. 4 is a perspective longitudinal section view of the dose sensing module,

FIG. 5 is a side view of a wiper assembly used in the dose sensing module,

FIG. 6 is a distal perspective view of the wiper assembly,

FIGS. 7 and 8 are respective examples of alternative wiper assemblies for use in the dose sensing module,

FIG. 9 is an exploded perspective view of a cartridge system according to another exemplary embodiment of the invention,

FIG. 10 is a side view of a proximal portion of the cartridge system of FIG. 9,

FIG. 11 is a perspective view of a guide element forming part of the cartridge system,

FIG. 12 is a longitudinal section view of the proximal portion of the cartridge system shown in FIG. 10,

FIG. 13 is a simplistic section view showing some dimensions of the cartridge system,

FIG. 14 is a simplistic section view showing a dose sensing module and a portion of the cartridge system during assembly,

FIG. 15 is a simplistic section view showing the dose sensing module in an assembled pre-use position in the cartridge system,

FIG. 16 is a cross-sectional view through section A-A of FIG. 15,

FIG. 17 is a longitudinal section view of a central portion of an injection device with the cartridge system and the dose sensing module,

FIGS. 18a and 18b are longitudinal section views of the guide element in respectively an undeformed and a deformed condition,

FIG. 19 is a different longitudinal section view of the central portion of the injection device, and

FIG. 20 is a cross-sectional view through section B-B of FIG. 19.

In the figures like structures are mainly identified by like reference numerals.

### DESCRIPTION OF EXEMPLARY EMBODIMENTS

When/If relative expressions, such as “upper” and “lower”, “left” and “right”, “horizontal” and “vertical”, “clockwise” and “counter-clockwise”, etc., are used in the following, these refer to the appended figures and not necessarily to an actual situation of use. The shown figures are schematic representations for which reason the configuration of the different structures as well as their relative dimensions are intended to serve illustrative purposes only.

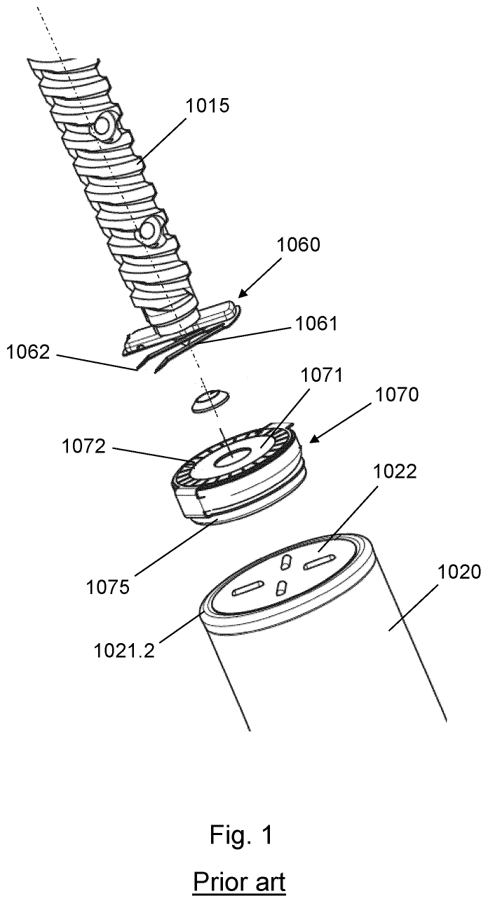

FIG. 1 shows a rotary sensor module according to the prior art, arranged between a distal end of a piston rod 1015 and a proximal end of a piston 1022 sealing a drug containing cartridge 1020 close to a proximal rim 1021.2 thereof. The sensor module, which is powered by a coin cell type battery 1075, comprises a first sensor part 1070 in the form of a flexible printed circuit board sheet having a proximally directed sensor surface 1071 on which 24 individual electrically conductive sensor areas 1072 are disposed circumferentially about a centre axis, and a second sensor part 1060 mounted on a distal end portion of the piston rod 1015 opposite the first sensor part 1070 and having contact structures in the form of two electrically connected flexible arms 1061, each terminating in a contact point 1062.

The first sensor part 1070 is adapted to engage, directly or indirectly, the piston 1022 such that no relative rotation therebetween is possible. The second sensor part 1060 is rotationally fixed to the piston rod 1015, and the contact points 1062 are adapted to engage and electrically connect various individual electrically conductive sensor areas 1072 upon relative rotational motion between the first sensor part 1070 and the second sensor part 1060, experienced as the piston rod 1015 rotates during a dose expelling action. This allows for an estimation of a total angular displacement exhibited by the piston rod 1015 during the dose expelling action and thereby of the amount of drug expelled.

As can be seen, even though the rotary sensor module is small-sized the transversal dimension of the first sensor part 1070 corresponds approximately to the internal diameter of the drug containing cartridge 1020. During assembly of the injection device incorporating the rotary sensor module, unless the individual components are completely aligned there is a significant risk that the first sensor part 1070 impacts the proximal rim 1021.2 of the drug containing cartridge 1020, causing fracture thereof. Obviously, if that happens the drug containing cartridge 1020 cannot be used and must be scrapped. This places severe demands on the tolerances in the assembly setup.

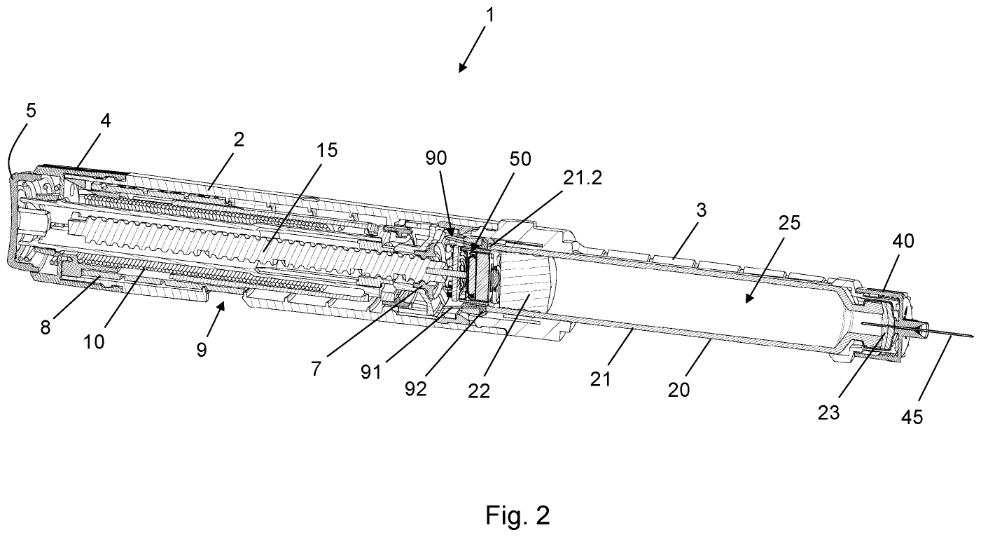

FIG. 2 is a perspective longitudinal section view of an injection device 1 having an integrated sensor module 50 for estimation of the size of an expelled dose of drug. The injection device 1 is of the prefilled autopen injector type, with an elongated housing 2 extending along a reference axis and accommodating a dose expelling mechanism. A cartridge holder 3, holding a cartridge 20 with an interior chamber 25 defined by a cartridge wall 21, a distal penetrable septum 23 and a proximal piston 22, is permanently fixed to the housing 2. The chamber 25 is at least substantially filled with a liquid substance (not visible). In the depicted state of the injection device 1 a needle assembly 40 is attached to a needle mount portion of the cartridge holder 3 in such a manner that an injection needle 45 has penetrated the septum 23 to establish fluid communication to the chamber 25.

A user operable dose dial 4 is arranged at a proximal end portion of the housing 2 for selective setting of a dose to be ejected from the cartridge 20. The dose dial 4 is operatively coupled with a scale drum 8 which displays a selected dose through a window 9. An injection button 5 is axially depressible to release a windable torsion spring 10. The release of the torsion spring 10 will cause a helical advancement of a piston rod 15 through a nut member 7 fixed in the housing 2 and thereby result in an execution of a dose expelling action.

Details of the dose setting and the dose expelling mechanisms are irrelevant to the present invention and will accordingly not be provided in the present text. For an example of how such mechanisms may be constructed reference is made to WO 2015/071354, particularly p. 10, I. 21-p. 15, I. 13. What is important is that the rotational movement of the piston rod 15 during dose expelling is correlated with the prompted movement of the piston 22 through the design of the piston rod thread and the nut member 7 such that a predetermined angular displacement of the piston rod 15 relative to the housing 2 corresponds to a predetermined axial displacement of the piston 22 relative to the cartridge wall 21. This relationship may in principle be chosen arbitrarily by the manufacturer, with a view to the dimensions of the cartridge 20. In the present example a 15° angular displacement of the piston rod corresponds to a specific axial displacement of the piston 22 which results in the expelling of 1 IU of the contained substance through the injection needle 45.

It is noted that the injection device 1 includes a guide element 90 having a funnel shaped guide body 91 and a circumferential seat 92. The circumferential seat 92 abuts a proximal rim 21.2 of the cartridge wall 21 defining a proximal opening of the cartridge 20. The guide element 90 and the cartridge 20 together constitute a cartridge system according to an embodiment of the present invention. By employing the whole cartridge system instead of just the cartridge 20 the strict requirements to radial alignment of the sensor module 50 with the proximal opening of the cartridge 20 are eased because the funnel shaped guide body 91 directs the sensor module 50 towards the proximal opening of the cartridge 20 during relative axial converging motion between the sensor module 50 and the cartridge 20 if the position of the sensor module 50 initially is somewhat radially offset. This will be discussed further below in connection with another exemplary embodiment of the invention.

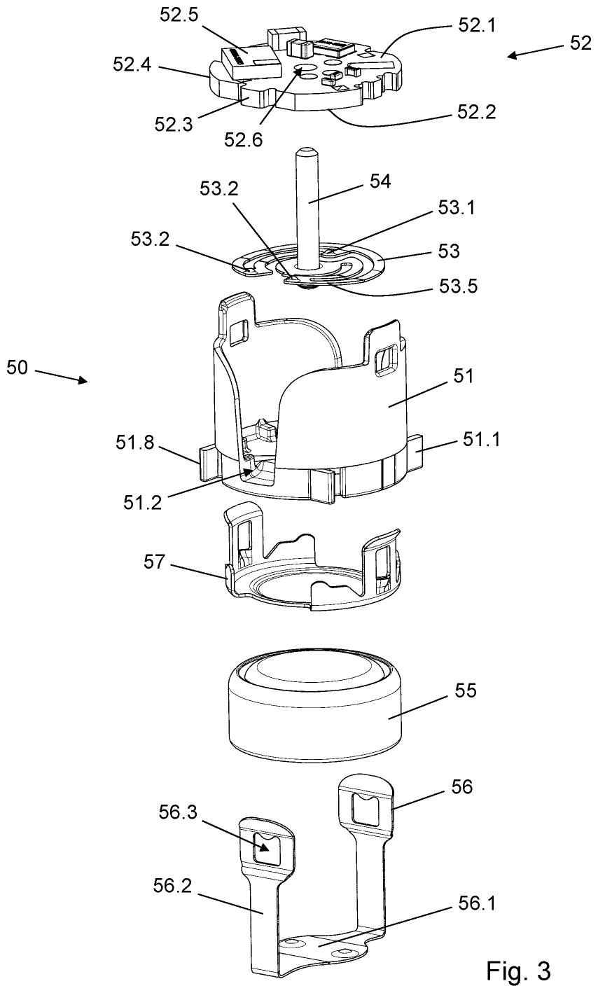

FIG. 3 is an exploded view highlighting the individual elements of the present sensor module 50. The sensor module 50 comprises a first sensor part in the form of a PCB assembly 52 with a rigid support sheet 52.4 having a proximal surface 52.1 carrying various electronic components 52.5, including a processor, and a distal surface 52.2 carrying a plurality of electrically conductive sensor areas (not visible), the configuration of which will be described below. The support sheet 52.4 has an overall circular periphery, but is provided with several notches, some of which resulting in a pair of diametrically opposite radial protrusions 52.3. Furthermore, the support sheet 52.4 has a central through-going bore 52.6.

The first sensor part is complemented by a second sensor part in the form of a wiper 53 being fixedly mounted to a piston rod connector 54 to ensure joint rotation therewith. The piston rod connector 54 extends axially through the through-going bore 52.6 and is adapted for press-fit engagement with a cavity in a distal end portion of the piston rod 15, as shown on FIG. 2. This provides for a joint movement of the piston rod 15 and the piston rod connector 54. The wiper 53 comprises one ground contact 53.1 and two code contacts 53.2 arranged on respective flexible arms 53.5 and adapted to galvanically connect with the electrically conductive sensor areas on the distal surface 52.2 of the support sheet 52.4, as described in more detail below. Notably, the ground contact 53.1 and the code contacts 53.2 are all proximally directed.

The two sensor parts, forming a rotary encoder system, are accommodated in a module housing 51 which also accommodates a power source in the form of a battery 55, a retainer 56 also functioning as a positive battery connector, and a rigid (negative) battery connector 57. The retainer 56 has a transversal support surface 56.1 for carrying the battery 55 and two axially extending opposite retainer arms 56.2. Each retainer arm 56.2 is provided with a proximal cut-out 56.3 shaped to receive one of the radial protrusions 52.3, thereby rotationally interlocking the retainer 56 and the PCB assembly 52 and axially restricting the support sheet 52.4. The module housing 51 has a pair of diametrically opposite side openings 51.2 shaped to receive the retainer arms 56.2 so as to rotationally interlock, or at least substantially rotationally interlock, the retainer 56 and the module housing 51, and a plurality of antirotation tabs 51.1 spaced apart along its circumference, each anti-rotation tab 51.1 comprising a contact surface 51.8 for interaction with an interior surface of the cartridge wall 21. The PCB assembly 52 is thus at least substantially rotationally locked with respect to the module housing 51, which in turn is rotationally frictionally fitted in the cartridge 20, which is rotationally fixed in the cartridge holder 3. The PCB assembly 52 is thereby at least substantially rotationally fixed with respect to the housing 2 and accordingly suitable as reference component for measuring angular displacements of the piston rod 15.

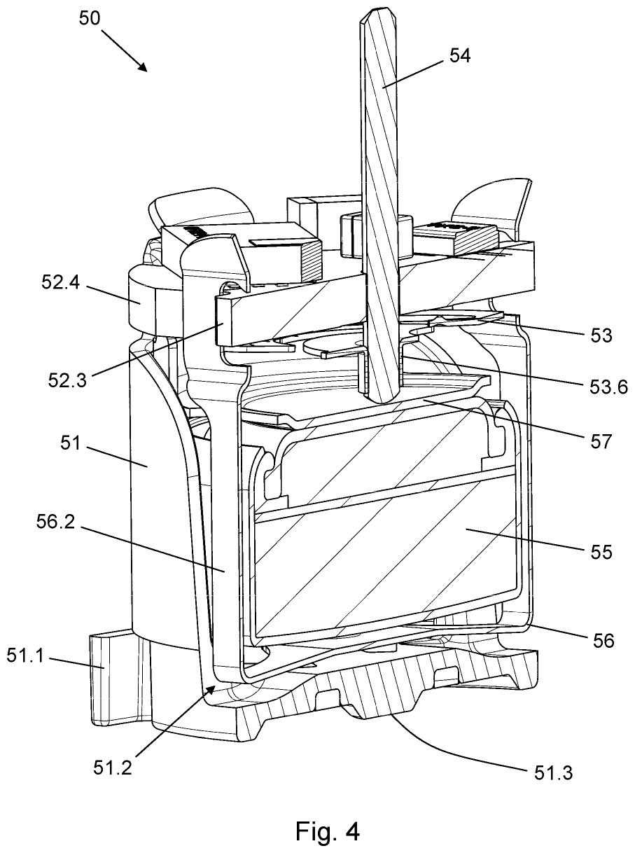

FIG. 4 is a perspective longitudinal section view of the sensor module 50 in an assembled state. As can be seen the piston rod connector 54 extends through the through-going bore 52.6 in the support sheet 52.4 and is press-fitted with a sleeve 53.6 on the wiper 53. The module housing 51 has a foot 51.3 which rests against the piston 22 (cf. FIG. 2). Furthermore, the figure shows the position of the retainer arms 56.2 in the side openings 51.2 and the arrangement of the radial protrusions 52.3 in the cut-outs 56.3. During a dose expelling action with the injection device 1 the rotation of the piston rod 15 is transferred to the piston rod connector 54 and further on to the wiper 53. The ground contact 53.1 and the code contacts 53.2 thus sweep the sensor areas of the distal surface 52.2 which remains, at least substantially, rotationally stationary due to the engagement between the radial protrusions 52.3 and the cut-outs 56.3, the fitting of the retainer arms 56.2 in the side openings 51.2, the frictional interface between the foot 51.3 and the piston 22, and the frictional interface between the anti-rotation tabs 51.1 and the cartridge wall 21.

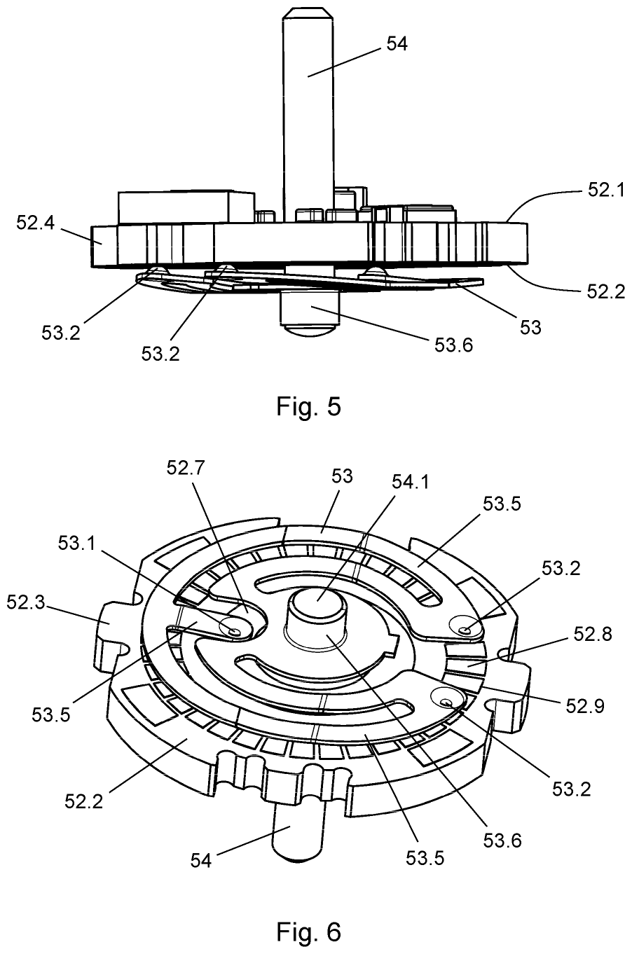

FIG. 5 is a side view of the two sensor parts showing the connection between the ground contact 53.1 and the code contacts 53.2 and the distal surface 52.2 of the support sheet 52.4, and FIG. 6 is a perspective distal view of the same. In the shown exemplary embodiment the aforementioned plurality of electrically conductive sensor areas on the distal surface 52.2 are arranged such that a single circular ground track 52.7 provides a ground connection for the ground contact 53.1 and 36 individual code fields 52.8 together constitute a code track 52.9 which the code contacts 53.2 are adapted to sweep. A secondary ground connection is provided through a spherical end 54.1 of the piston rod connector 54 contacting the (negative) battery connector 57. The secondary ground connection may be relevant to stabilise the signal output in case the dynamics of the dose expelling mechanism generates vibrations in the sensor module 50.

As the piston rod connector 54 rotates jointly with the piston rod 15 during a dose expelling action the two code contacts 53.2, which are circumferentially separated by 45°, respectively sweep the code track 52.9, generating signals representative of the angular position of the wiper 53 as different code fields 52.8 get connected to ground. The two sensor parts output a 4-bit Gray code, i.e. eight different codes which for a 360° rotation of the wiper 53 are repeated nine times, giving 72 distinguishing codes. This output thus forms the basis for an estimation, by one or more of the electronic components 52.5 including the processor, of the total angular displacement of the piston rod 15 during a dose expelling action, and thereby for an estimation of the expelled dose.

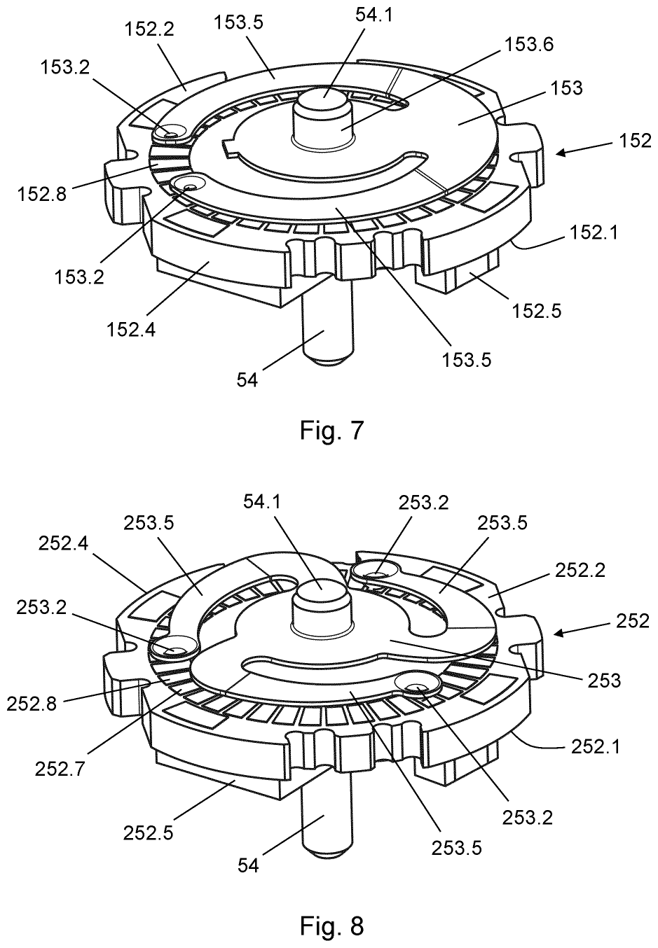

FIG. 7 is a perspective distal view of two sensor parts of an alternative rotary encoder system which may be employed in lieu of the one described above. The sensor parts comprise a wiper 153 and a PCB assembly 152 held in mutual position by the piston rod connector 54 in a manner similar to that disclosed in connection with the previous embodiment. The geometrical configuration of the PCB assembly 152 as well as its interaction with other components of the sensor module is identical to that of the formerly described PCB assembly 52. Particularly, the PCB assembly 152 comprises a rigid support sheet 152.4 having a proximal surface 152.1 which carries various electronic components 152.5, including a processor, and a distal surface 152.2 on which is disposed a plurality of electrically conductive code fields 152.8 arranged side by side to thereby provide a circular code track. However, contrary to the former embodiment the distal surface 152.2 does not comprise a dedicated ground track. Instead, the ground connection is supplied via the spherical end 54.1 of the piston rod connector 54 being in contact with the (negative) battery connector 57, similarly to the above described.

The wiper 153 comprises a sleeve 153.6 press-fitted onto the piston rod connector 54, to ensure joint rotation of the piston rod 15 and the wiper 153, and two code contacts 153.2, each arranged at an end portion of a flexible arm 153.5 capable of axial deflection. The code contacts 153.2 are angularly separated by 45° and will when rotated relative to the distal surface 152.2 sweep the code fields 152.8 and produce a 4-bit Gray code, similarly to the previous embodiment.

FIG. 8 is a perspective distal view of two sensor parts of another alternative rotary encoder system. Similarly to the previous embodiments the sensor parts comprise a wiper 253 and a PCB assembly 252 held in mutual position by the piston rod connector 54. The geometrical configuration of the PCB assembly 252 as well as its interaction with other components of the sensor module is identical to that of the formerly described PCB assembly 52. Particularly, the PCB assembly 252 comprises a rigid support sheet 252.4 having a proximal surface 252.1 which carries various electronic components 252.5, including a processor, and a distal surface 252.2 on which is disposed a plurality of electrically conductive sensor areas.

However, contrary to the former embodiments the distal surface 252.2 carries 40 electrically conductive sensor areas arranged in a circular track pattern where every other sensor area constitutes a ground field 252.7 and every other sensor area constitutes a code field 252.8. A secondary ground connection is supplied via the spherical end 54.1 of the piston rod connector 54 being in contact with the (negative) battery connector 57, as described above in connection with the first embodiment of the invention.

A wiper 253 is attached to the piston rod connector 54 and is adapted to sweep the 40 electrically conductive sensor areas as the piston rod 15 rotates during a dose expelling action (as described above). The wiper 253 has three flexible arms 253.5, each terminating in a contact point 253.2 which is adapted to galvanically connect with a ground field 252.7 or a code field 252.8, depending on the angular position of the wiper 253 relative to the PCB assembly 252. The three contact points 253.2 are separated 120° from each other such that one contact point 253.2 is always connected to a ground field 252.7 and two contact points 253.2 are always connected to a code field 253.8. The two sensor parts output a 4-bit Gray code and offer a higher resolution than the former two embodiments of the invention, enabling an even more accurate estimation of the total relative angular displacement between the PCB assembly 252 and the wiper 253, and thereby of the total angular displacement of the piston rod 15 relative to the housing 2, during a dose expelling event.

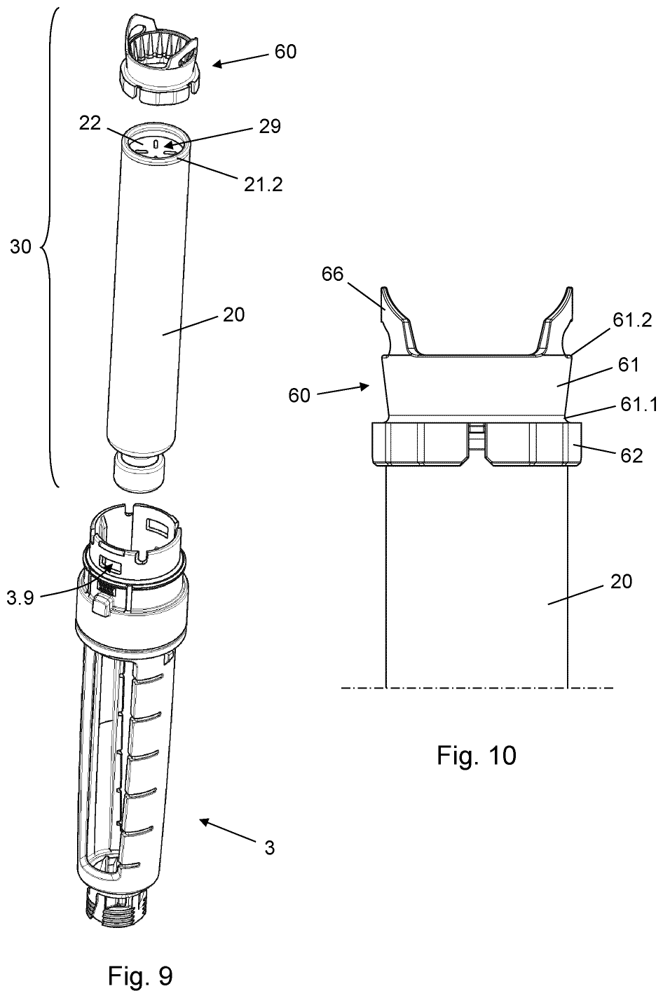

FIG. 9 is an exploded view of a cartridge system 30 according to another exemplary embodiment of the invention together with the cartridge holder 3. The cartridge system 30 comprises the drug cartridge 20 which is sealed by the piston 22 an axial distance from the proximal rim 21.2, whereby an outer cavity 29 is formed between the piston 22 and the proximal rim 21.2, and a guide element 60. The cartridge holder 3 is configured to receive the drug cartridge 20, as shown in FIG. 2, and comprises openings 3.9 for fixation to the housing 2 of the injection device 1.

In FIG. 10 a proximal portion of the cartridge system 30 is depicted. The guide element 60 is a single piece component made of low-density polyethylene (LDPE) and comprises a main guide body 61, and a guide collar 62 configured to engage the proximal rim 21.2 of the cartridge 20. The main guide body 61 extends axially between a distal guide body end 61.1 and a proximal guide body end 61.2, and the distal guide body end 61.1 borders the guide collar 62. A pair of diametrically opposite flanges 66 extend proximally from the proximal guide body end 61.2. The purpose of these flanges 66 will be clear from the below.

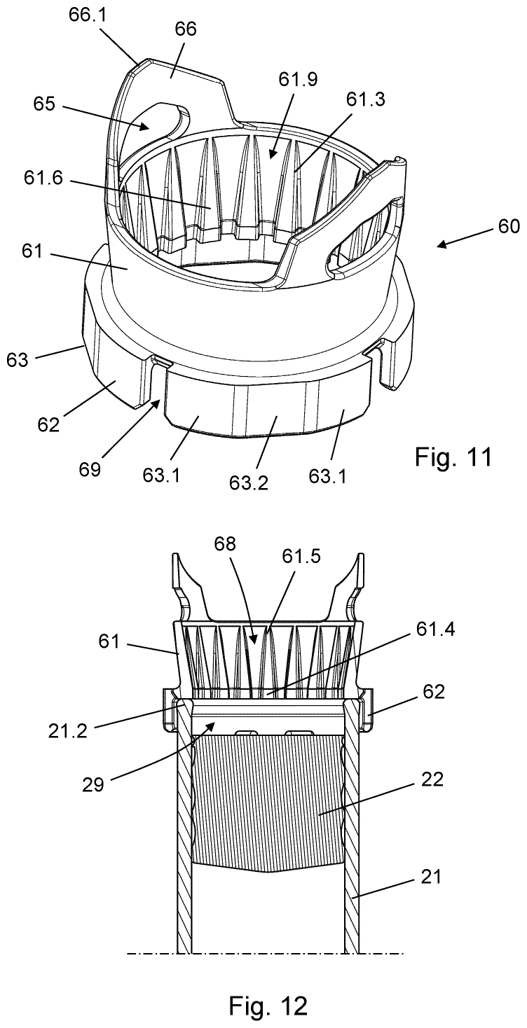

FIG. 11 is a perspective view of the guide element 60, revealing several constructional details. Firstly, the guide collar 62 is divided into four identical collar partitions 63, each of which is circumferentially spaced apart from a neighbouring collar partition, thereby providing four evenly distributed collar openings 69 along the guide collar circumference. Each collar partition 63 comprises two convex flank sections 63.1 separated by a concave central section 63.2.

Secondly, the main guide body 61 has an interior surface which is provided with a plurality of evenly distributed axially extending splines 61.3 having respective radially facing surfaces 61.6 for guiding a sensor module. The splines 61.3 taper both radially and circumferentially towards the proximal guide body end 61.2. Two neighbouring splines 61.3 define an intermediate keyway 61.9 which accordingly tapers circumferentially towards the distal guide body end 61.1.

Finally, each flange 66 has a shape which resembles a handle, with a central hole 65 and an obtuse apex 66.1. This particular shape of the flanges 66 along with the relatively soft polymer material render them axially elastically compressible.

FIG. 12 is a longitudinal section view of the proximal portion of the cartridge system 30, from which it can be seen that each spline 61.3 extends between a distal spline end 61.4 at the distal guide body end 61.1 and a proximal spline end 61.5 at the proximal guide body end 61.2, and that the distal spline end 61.4 is markedly wider circumferentially than the proximal spline end 61.5. Also, the radially facing surfaces 61.6 of the splines 61.3 together constitute a funnel-shaped internal guide surface which tapers radially towards the distal guide body end 61.1. A wider inlet 68 for the sensor module is thus provided at the proximal guide body end 61.2. Furthermore, the guide collar 62 surrounds a proximal end portion of the cartridge wall 21, including the proximal rim 21.2.

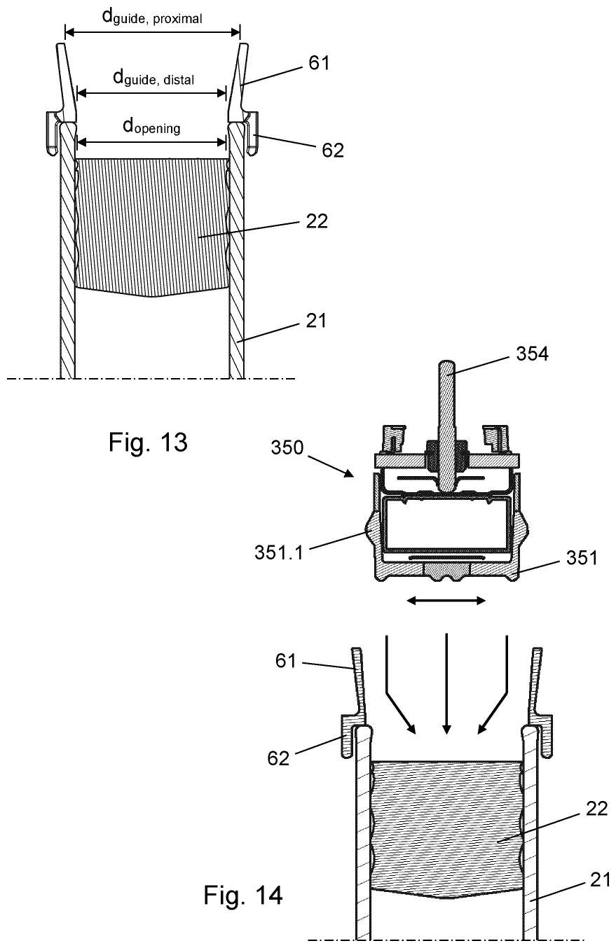

For the sake of clarity, FIGS. 13-15 are simplistic section views in the sense that only the material present in the specific sections is visible.

Hence, FIG. 13 is a simplistic longitudinal section view of the proximal portion of the cartridge system 30, illustrating some important radial dimensions. The section is cut through a pair of opposite splines 61.3 and therefore shows the diameter of the funnel-shaped internal guide surface of the main guide body 61, which decreases gradually towards the proximal opening of the cartridge 20 from a proximal guide diameter, d_{guide, proximal}, at the proximal guide body end 61.2 to a distal guide diameter, d_{guide, distal}, at the proximal guide body end 61.1. The distal guide diameter, d_{guide, distal}, corresponds to the diameter of the proximal opening of the cartridge 20, d_{opening}.

FIG. 14 is a simplistic section view of a sensor module 350 in a pre-assembly position outside the cartridge system 30. The structure of the sensor module 350 resembles that of the previously described sensor module 50. Accordingly, the sensor module 350 comprises a module housing 351 with a foot for engagement with the piston 22, and a piston rod connector 354 for engagement with the piston rod (not shown). The main difference vis-à-vis the former sensor module 50 is that the module housing 351 comprises a pair of anti-rotation tabs 351.1 which are arranged more proximally than the anti-rotation tabs 51.1 of the module housing 51. The anti-rotation tabs 351.1 are radially inwardly deflectable against a radial restoration force to allow passage through the proximal opening of the cartridge 20 and subsequent firm connection with the cartridge wall 21.

Contrary to the section in FIG. 13 the section in FIG. 14 is cut through a pair of opposite keyways 61.9, and the taper of the main guide body 61 accordingly appears smaller in this view, just as the guide collar 62 does not seem to cover the proximal rim 21.2 entirely. This section is presented to illustrate the relative angular orientations of the sensor module 350 and the cartridge system 30 during assembly, where the anti-rotation tabs 351.1 are aligned with respective keyways 61.9 for sliding reception therein.

As is indicated by the arrows in FIG. 14 the tapering internal guide surface of the main guide body 61 allows for some play in the assembly setup in that minor radial misalignments of the sensor module 350 with the proximal opening of the cartridge 20 will be compensated during the relative axial converging motion between the sensor module 350 and the cartridge 20 such that the sensor module 350 eventually enters the outer cavity 29 safely without impacting the proximal rim 21.2.

In fact, because of the circumferential tapering of the splines 61.3 forming keyways 61.9 that are wider at the proximal guide body end 61.2 than at the distal guide body end 61.1 the sensor module 350 need not even initially be in strict angular alignment with the guide element 60, as the keyways 61.9 will receive the anti-rotation tabs 351.1 at a wider angle on entry into the main guide body 61 and subsequently guide the anti-rotation tabs 351.1 into a proper angular orientation as the sensor module 350 approaches the outer cavity 29.

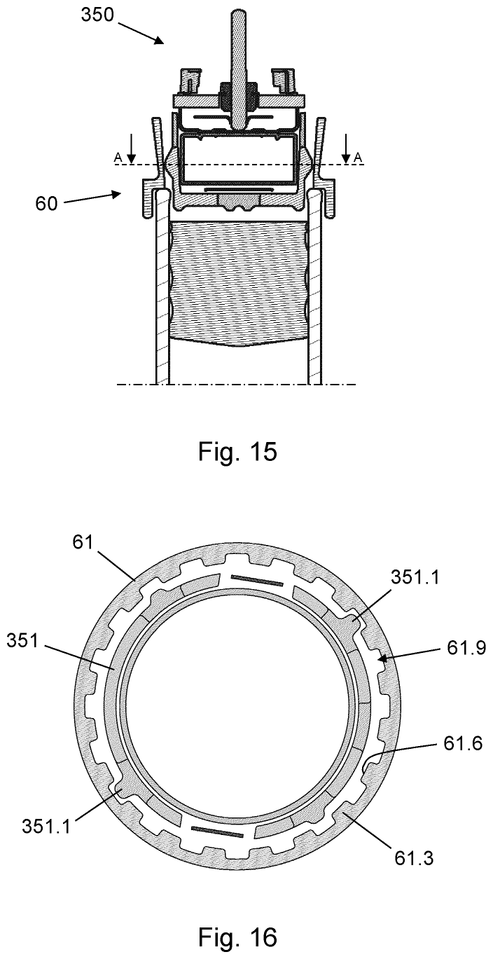

FIG. 15 shows the sensor module 350 in an assembled pre-use position in the cartridge system 30. In this pre-use position the module housing 351 is axially spaced apart from the piston 22 and each anti-rotation tab 351.1 is unstrained and rests in a narrow section of a keyway 61.9.

FIG. 16, which is a cross-sectional view through section A-A, shows that at this point the antirotation tabs 351.1 are firmly engaged with the respective keyways 61.9, and the module housing 351 is accordingly rotationally locked with respect to the guide element 60.

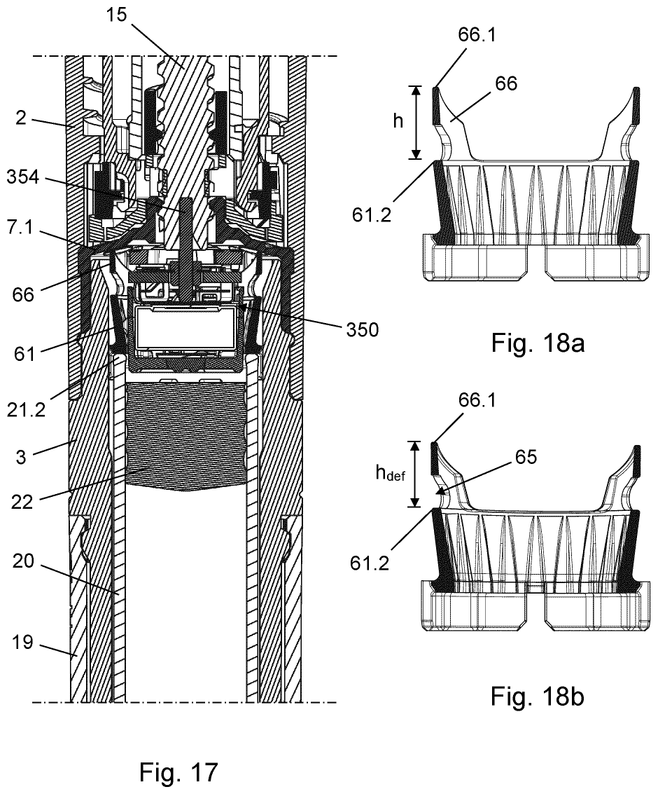

In the pre-use position of the sensor module 350 the piston rod connector 354 is prevented from rotating about the longitudinal axis, because the piston rod 15 (see FIG. 17) is rotationally fixed with respect to the housing 2 in a pre-use state of the injection device 1. Furthermore, the module housing 351 is prevented from rotating because the anti-rotation tabs 351.1 engage with the keyways 61.9, and the guide element 60 is rotationally fixed with respect to the housing 2 (which will be explained further below).

The sensor module 350 is thus rotationally fixed in a pre-use state of the injection device 1, so even if the injection device 1 is dropped on the ground or otherwise exhibits jolting movements, e.g. in connection with transportation or general handling, there is no risk of prematurely wakening the sensor electronics and thereby draining the battery.

The sensor module 350 is adapted to be displaced axially, during the first use of the injection device, from the pre-use position to an in-use position in the outer cavity 29. During this displacement from the pre-use position to the in-use position the anti-rotation tabs 351.1 will be deflected radially inwardly against the radial restoration force provided by the structure of the module housing 351, and the sensor module 350 accordingly transitions from an unstrained state to a strained state. Once the anti-rotation tabs 351.1 have passed the proximal rim 21.2 they will apply a radially outwardly directed force to, and thus increase friction in the interface with, the cartridge wall 21, thereby impeding rotation of the module housing 351 relative to the cartridge 20. It is advantageous to shelve the injection device 1 with the sensor module 350 in the unstrained state to avoid the risk of strained anti-rotation tabs 351.1 losing tension over time, as this would lead to a reduction of the contact force, and resultantly loss of friction, in the interface with the cartridge wall 21.

FIG. 17 is a longitudinal section view of a central portion of the injection device 1 with the cartridge system 30 and the sensor module 350 incorporated. The injection device is shown in a pre-use state where the sensor module 350 is in the pre-use position and a protective cap 19 is attached to the cartridge holder 3. Notably, the guide element 60 is located between the cartridge 20 and a transversal nut portion 7.1 of the nut member 7, the apex 66.1 of each flange 66 abutting a distally facing surface of the transversal nut portion 7.1. The cartridge 20 is thus elastically supported between the cartridge holder 3 and the nut member 7 due to the axial compressibility of the flanges 66. Thereby, the cartridge wall 21 is protected from shock damages resulting from the injection device 1 being dropped or otherwise roughly handled, as the flanges 66 will cushion any axial displacement of the cartridge 20 relative to the housing 2 and the nut member 7.

FIGS. 18a and 18b are longitudinal section views of the guide element 60 illustrating the cushioning effect as the flanges 66 deform during an axial impact. The flanges 66 exhibit an initial axial height, h, in an undeformed state of the guide element 60, shown in FIG. 18a, and a smaller axial height, h_{def}, in a deformed state of the guide element 60, shown in FIG. 18b, where the cartridge system 30 is transiently exposed to a significant axial force. The flanges 66 deform elastically in response to the proximal rim 21.2 applying an axial compressive force to the guide collar 62, whereby the respective central holes 65 are temporarily flattened. Immediately after the impact the flanges 66 recover to the initial axial height, h, and thereby return the cartridge 20 to the position shown in FIG. 17.

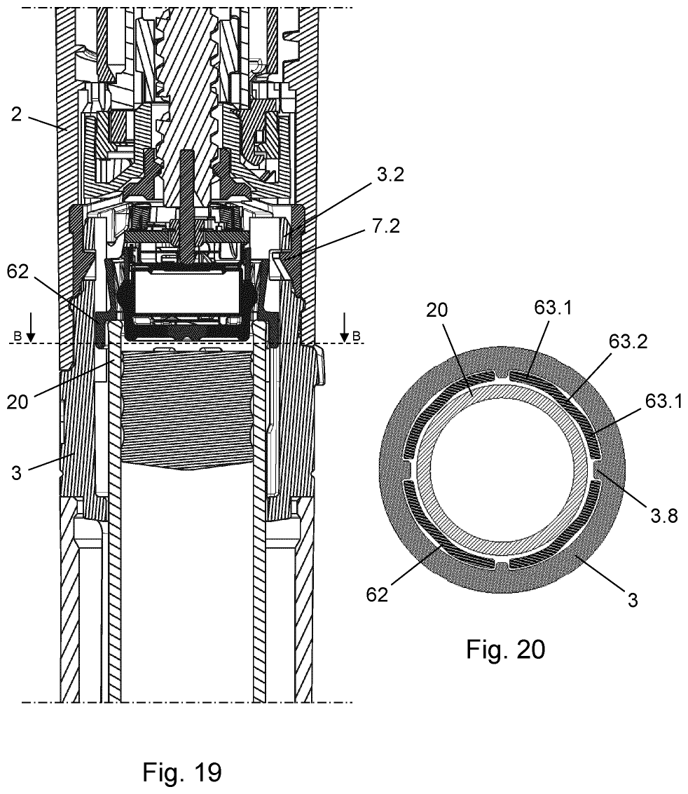

FIG. 19 depicts the central portion of the injection device 1 in a different longitudinal section view, from which it is seen that a top portion 3.2 of the cartridge holder 3 is positioned proximally of a pair of opposite radially inwardly projecting snaps 7.2 of the nut member 7. During assembly of the injection device 1 the top portion 3.2, which possesses a certain radial flexibility, is urged past the snaps 7.2, whereby the snaps 7.2 respectively enter the openings 3.9 (see FIG. 9) to provide a both translationally and rotationally interlocked connection between the housing 2 and the cartridge holder 3.

FIG. 20 is a cross-sectional view through section B-B, albeit (for the sake of clarity) not showing the housing 2. This figure shows the collar openings 69 occupied by respective internal protrusions 3.8 on the cartridge holder 3, providing a rotationally interlocked connection between the guide element 60 and the cartridge holder 3. As a consequence, since the cartridge holder 3, as explained above, is rotationally fixed with respect to the housing 2, so is the guide element 60. The guide element 60 thus provides rotational fixation of the module housing 351 relative to the housing 2 in the shown pre-use position of the sensor module 350.

The respective collar partitions 63 offer an additional shock absorption in connection with potential radial impacts to the injection device 1, such as if the injection device 1 is dropped to one side. The alternating convex flank sections 63.1 and concave central section 63.2 of each collar partition 63 together with the air gaps resultantly formed between the convex flank sections 63.1 and the cartridge 20 and between the concave central section 63.2 and the cartridge holder 3 provoke a spring-like action and thereby a cushioning effect which protects the proximal end portion of the cartridge wall 61, including the proximal rim 21.2.

1. A cartridge system for use in a drug delivery device, comprising:
a drug cartridge comprising
a cartridge body extending along a reference axis between a distal outlet end portion and a proximal rim, and
a displaceable piston arranged in the cartridge body an axial distance from the proximal rim, an outer cavity thus being formed between the displaceable piston and the proximal rim, and
a guide element comprising
a main guide body, and
a rim interface member adapted to abut or engage the proximal rim and thereby cover the proximal rim at least partially,
wherein the main guide body extends between a first main guide body end bordering the rim interface member and a second main guide body end and defines a passage for a sensor unit.

2. A cartridge system according to claim 1, wherein the main guide body comprises an interior guide surface configured to guide the sensor unit into the outer cavity, and wherein the interior guide surface tapers radially towards the first main guide body end.

3. A cartridge system according to claim 1, wherein the main guide body comprises a plurality of splines extending axially between the first main guide body end and the second main guide body end and a plurality of intermediate keyways formed by the plurality of splines each of the plurality of splines comprising a radially facing surface for guiding the sensor unit into the outer cavity.

4. A cartridge system according to claim 3, wherein each of the plurality of splines tapers radially towards the second main guide body end.

5. A cartridge system according to claim 3, wherein each of the plurality of splines tapers circumferentially towards the second main guide body end.

6. A cartridge system according to claim 1, wherein the rim interface member comprises a circumferential collar adapted to surround a proximal exterior end portion of the cartridge body, the circumferential collar having alternating convexly and concavely shaped sections.

7. A cartridge system according to claim 1, wherein the rim interface member comprises a circumferential collar adapted to surround a proximal exterior end portion of the cartridge body, the circumferential collar comprising a plurality of collar partitions, each collar partition being circumferentially spaced apart from a neighbouring collar partition, providing respective collar openings therebetween.

8. A cartridge system according to claim 7, wherein each collar partition comprises at least one convexly shaped section and at least one concavely shaped section.

9. A cartridge system according to claim 7, further comprising a cartridge holder for accommodating the drug cartridge, wherein the cartridge holder comprises a radially inwardly extending protrusion adapted to be received in one of the collar openings, thereby rotationally interlocking the cartridge holder and the guide element.

10. A cartridge system according to claim 1, wherein the guide element further comprises a plurality of axially compressible flange members extending axially from the second main guide body end.

11. A cartridge system according to claim 10, wherein the plurality of axially compressible flange members constitutes two axially compressible flange members arranged diametrically opposite one another.

12. A guide element for use in a cartridge system according to claim 1.

13. A drug delivery device comprising a cartridge system according to claim 1.

14. A drug delivery device according to claim 13, further comprising:
a housing,
a dose expelling mechanism comprising an axially advanceable piston rod, and
a sensor unit for determining a size of an expelled dose, arranged at least partially in the outer cavity and comprising a proximal module part rotationally locked with respect to the axially advanceable piston rod and a distal module part abutting the displaceable piston.

15. A drug delivery device comprising:
a cartridge system according to claim 10,
a housing,
a dose expelling mechanism comprising an axially advanceable piston rod, and
a sensor unit for determining a size of an expelled dose, arranged at least partially in the outer cavity and comprising a proximal module part rotationally locked with respect to the axially advanceable piston rod and a distal module part abutting the displaceable piston,
wherein each of the plurality of axially compressible flange members abuts a transversally extending interior portion of the housing, or a transversally extending structure axially fixed with respect to the housing.
